(19) United States
(12) Patent Application Publication
(10) Pub. No.: US 2003/0059071 A1
(43) Pub. Date: Mar. 27, 2003

(54) Personal audio device with hearing protection

(76) Inventor: John Dunham, Reno, NV (US)

Correspondence Address: KLARQUIST SPARKMAN, LLP, 121 SW SALMON STREET, SUITE 1600, PORTLAND, OR, 97204, US

(21) Appl. No.: 10/254,997

(22) Filed: Sep. 24, 2002

Related U.S. Application Data
(60) Provisional application No. 60/323,847, filed on Sep. 24, 2001.

(51) Int. Cl. A61F 11/06; H04R 1/10; H04R 5/02

(52) U.S. Cl. 381/309; 381/72; 381/74

(57) ABSTRACT

An integrated personal audio device that provides hearing protection is disclosed. The device includes a flexible band configured to extend at least partially around a head of a user. An audio transmitter is coupled to the band. The band has two legs that terminate at respective end portions. Each end portion is coupled to an audio speaker, which is electrically connected to the audio transmitter. A hearing protector is mounted onto each respective audio speaker. Each hearing protector has a sound channel configured to transmit sound from the respective audio speaker to the user's ear. The sound channel may comprise a Venturi-shaped chamber. The sound channel may, for instance, include: (1) a sound-expansion-chamber portion; (2) a central-chamber portion having a smaller diameter than the sound-expansion-chamber portion; and (3) an end-chamber portion having a larger diameter than the central-chamber portion.

### RELATED APPLICATIONS

This application claims the benefit of U.S. Provisional Application No. 60/323,847 filed Sep. 24, 2001.

### FIELD OF INVENTION

This disclosure relates to a personal audio device that also provides hearing protection to the user.

### BACKGROUND

Personal audio devices typically combine a compact audio transmitter with headphones and allow a user to enjoy the benefits of an audio sound system without sacrificing mobility and convenience. One of the better known types of personal audio devices consists of an audio transmitter connected to a pair of earphones through a wire that hangs freely between the two components. Examples of this type of device include the Walkman® series of devices produced and marketed by Sony Corporation. Because the audio transmitter is separate from the earphones, however, the user must typically hold or attach the receiver/transmitter to his body, often in an uncomfortable or awkward position. Further, because the wire hangs freely between the two units, it may interfere with the free movement of the user. This can be irritating and potentially dangerous to users, whose full range of motion is impaired.

Another type of personal audio device consists of an audio transmitter integrated with earphones into a headset worn by the user. Examples include the Panasonic SV-SD05 audio player headset device and the device described in U.S. Pat. No. 6,091,832. Because these units are typically designed primarily for comfort, they fit rather loosely on the user's head and are unsuitable for active use. Additionally, the headsets do not offer the user any hearing protection, and thus are unsuitable for users in loud environments.

Yet another type of personal audio device combines an audio headset with ear-muff-style hearing protectors. Examples of this type of device include the communication ear muffs sold by Elvex Corp., the aviation headsets sold by Peltor Corp., and the communications device described in U.S. Pat. No. 5,426,719. The ear muffs of this type of device fit entirely around the user's ears, thus blocking noise from the surrounding environment, and contain micro-speakers that transmit sound to the user's ears. Although this type of personal audio device provides hearing protection, the device is typically expensive, bulky, and heavy. Accordingly, the devices are unsuitable for active use or for convenient storage and transportation.

Like personal audio devices, hearing protective devices that do not have an audio transmitter are also available in a variety of types and sizes. One such device, for instance, is the band earplug. Examples of band earplugs include the devices described in U.S. Pat. Nos. 4,490,857, 5,298,691, and 5,824,966. Band earplugs generally consist of a headband with resilient, foam hearing protectors attached at the ends of the band. The band is constructed of a flexible, plastic material and is designed to hold the hearing protectors firmly against the user's ears. Unlike the personal audio devices described above, however, the hearing protectors of this type of device are designed solely for hearing protection and are not designed to permit the passage of any sound.

None of the above inventions provide the user with a low-cost, lightweight personal audio device that provides hearing protection.

### SUMMARY

In view of the shortcomings of the conventional devices described above, an integrated personal audio device that also provides hearing protection is described herein.

In one implementation, the device includes a flexible band configured to extend at least partially around a head of a user. The band includes two legs that terminate at respective end portions that are located adjacent the user's ears during use. An audio transmitter is coupled to the band. The audio transmitter may be located approximately midway between the end portions of the band. Moreover, the audio transmitter may comprise any suitable audio transmitting device (e.g., a radio receiver, an MP3 player, etc.). The audio transmitter may also include a jack for inputting audio signals from an external audio source. An audio speaker is attached to each of the respective end portions of the band. The audio speakers are electrically connected to the audio transmitter by wires, which may be integrally formed into the band. A hearing protector is mounted onto each of the respective audio speakers and has a sound channel configured to transmit sound from the speaker to the ear of the user. The band of the device may have a resiliency sufficient to urge the hearing protectors against the ears of the user to at least partially engage the hearing protectors against or in the ear canals of the user. When the device is worn by the user, the hearing protectors attenuate sound relative to the user (e.g., sound may be attenuated to a sound level of 90 db or below).

The hearing protectors may be constructed of compressible foam, silicon rubber, or a suitable hypoallergenic rubber compound. The hearing protectors may also have a main body portion and an adjacent tapered portion. In one implementation, the hearing protectors form supra-aural hearing protectors, wherein the tapered portion of the protector does not enter the ear canal of the user. In other implementations, however, the hearing protectors form semi- or inner-aural hearing protectors, wherein the tapered portion of the protector does enter the ear canal of the user. The hearing protectors may also include a speaker chamber formed in the main body portion and configured to receive an audio speaker and a part of the end portion. The hearing protectors may be removable from the audio speakers. Therefore, the user can easily replace soiled or old hearing protectors with new ones. Alternatively, the user can replace the sound-transmitting hearing protectors with non-sound-transmitting hearing protectors, which are adapted to fit the headband and provide the user with traditional hearing protection without any audio transmission.

The sound channel of the hearing protectors may comprise a Venturi-shaped channel between the speaker and an end of the tapered portion. The sound channel may, for instance, include: (1) a frustoconical sound-expansion-chamber portion adjacent the speaker chamber; (2) a cylindrical central-chamber portion adjacent the sound-expansion-chamber portion; and (3) a frustoconical end-chamber portion adjacent the central-chamber portion and extending to the end of the tapered portion. The central-chamber portion may have a smaller diameter than the sound-expansion-chamber portion, and the end-channel portion may have a larger diameter than the central-chamber portion. Moreover, the walls of the end-channel portion may have a flared or trumpet shape.

The disclosed device is economical and lightweight and may be utilized by users in a number of different arenas. For instance, spectators at automobile races may use the device to listen to a radio broadcast of the race while protecting their ears from the harmful noise of the event. Workers in noisy environments may use the device to protect their hearing while enjoying an audio broadcast. Runners or other active athletes may use the device during a workout or race.

The foregoing and additional features and advantages of the disclosed technology will be more readily apparent from the following detailed description, which proceeds with reference to the accompanying drawings.

### BRIEF DESCRIPTION OF THE DRAWINGS

FIG. 1 is a front perspective view of an implementation of the personal audio device.

FIG. 2 is a back view of a portion of the personal audio device of FIG. 1, showing the audio transmitter.

FIG. 3 is an exploded perspective view showing the construction of the audio transmitter of FIG. 1.

FIG. 4 is a right side view of the personal audio device shown in FIG. 1.

FIG. 5 is another perspective view of the device of FIG. 1, showing a user and various exemplary positions in which the device can be worn.

FIG. 6 is a cross-sectional side view of a first implementation of a hearing protector used with the personal audio device.

FIG. 7 is a cross-sectional side view of a second implementation of a hearing protector used with the personal audio device.

### DETAILED DESCRIPTION

Disclosed below are representative implementations that are not intended to be limiting in any way.

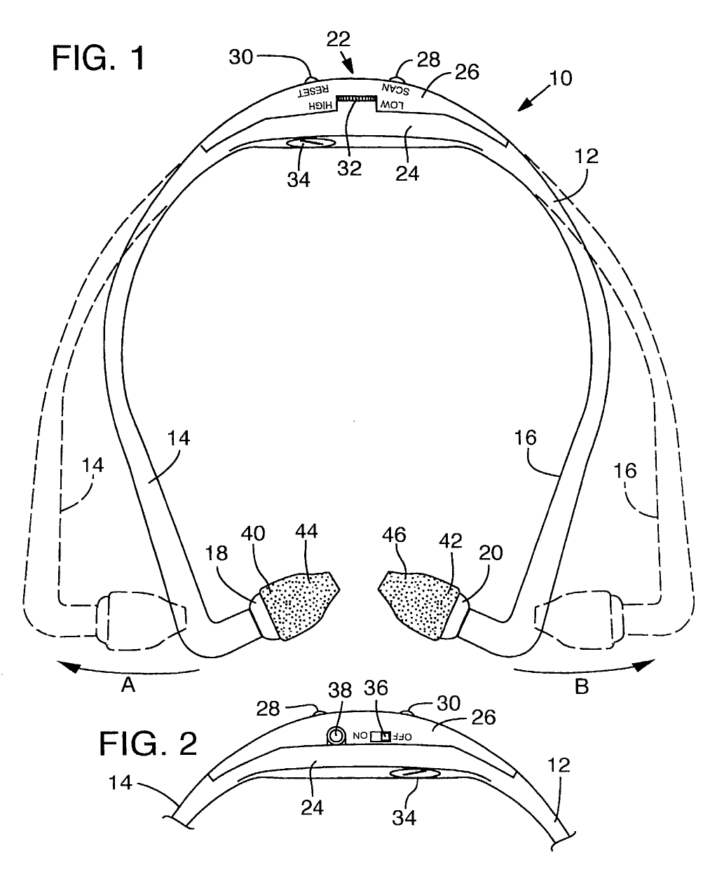

FIG. 1 shows a front view of a personal audio device 10 according to a representative implementation. The personal audio device 10 includes a band 12 having two legs 14, 16 that extend at least partially around the head of a user and terminate at two respective end portions 18, 20 of the band. The band 12 is designed so that each of the end portions 18, 20 interfaces with the ears of the user while the band is being worn. The dimensions of the band 12 may vary depending on the intended user (e.g., average adult, child, etc.). The band 12 is generally made from a flexible, resilient material (e.g., plastic, polyethylene, polypropylene, etc.) and may be constructed using well-known molding techniques (e.g., injection molding). The band 12 may have a first unflexed shape when it is not being worn by a user (shown by the solid lines) and a second flexed shape when the legs 14, 16 are bent outward to fit at least partially around a user's head (shown by the dotted lines).

The device 10 further includes an audio transmitter 22 coupled to the band 12. In the illustrated implementation, the audio transmitter 22 is integrated with the band 12 within a smoothly contoured enclosure formed in the band. Although the audio transmitter 22 shown in FIG. 1 is centrally positioned on the band 12, the audio transmitter may be located at various other positions on the band (e.g., adjacent an end portion). Further, the audio transmitter 22 may comprise multiple units that together perform the function of a single audio transmitter. The audio transmitter 22 may include RF shielding to filter radio interference associated with nearby electrical equipment. An antenna (not shown) may be connected to the audio transmitter 22. The antenna may be embedded in one or both of the legs 14, 16. Generally, the antenna will consist of a thin wire integrated into the band 12, but the antenna may be contained internally within the audio transmitter 22 or may be attached externally through a jack 38 (discussed below).

The audio transmitter 22 shown in FIG. 1 is positioned between a support portion 24 and a cover portion 26 of the band 12. In the illustrated implementation, the audio transmitter 22 is a battery-powered digital radio receiver. A scan button 28 and a reset button 30 may be used for tuning the audio transmitter 22. Specifically, depression of the reset button 30 causes the radio receiver to tune to the lowest available radio frequency (e.g., 87.5 MHz). Depression of the scan button 28 then advances the tuning to the next available radio frequency (e.g., 88.7 MHz). Because the illustrated radio receiver only has two buttons used for tuning, it provides the user with a simple tactile means of adjusting the tuning while the device 10 is being worn. A volume wheel 32 may be used to adjust the volume of the audio produced by the audio transmitter 22. A battery cover 34 for enclosing a battery (e.g., a round coin-cell battery) may be located on the lower, generally flat surface of the support portion 24.

Although a radio is shown in FIG. 1, the audio transmitter 22 may comprise any device that produces electric audio transmissions. For example, the audio transmitter 22 may be an MP3, CD, MiniDisk, or satellite radio audio source. Moreover, although only one particular set of controls is shown in FIG. 1, there are a variety of controls or modifications that may be made to the audio transmitter 22. For instance, the audio transmitter 22 may include a display (e.g., an LED or LCD display) that shows information related to the audio being transmitted (e.g., the frequency of the radio station to which the radio is currently tuned). Similarly, if the audio transmitter 22 is an MP3 device, the controls may include play, skip, scan, and stop buttons.

Turning briefly to FIG. 2, which shows an opposite side of the audio transmitter 22, a power switch 36 (e.g., a button or switch) and a jack 38 may be provided. The jack 38 is an input to the audio transmitter 22 used to electrically couple the audio transmitter with an external device (e.g., an external CD player, MP3 player, radio, etc.). Thus, audio from an external source may be input into the personal audio device 10. The jack 38 may also operate as an external antenna input.

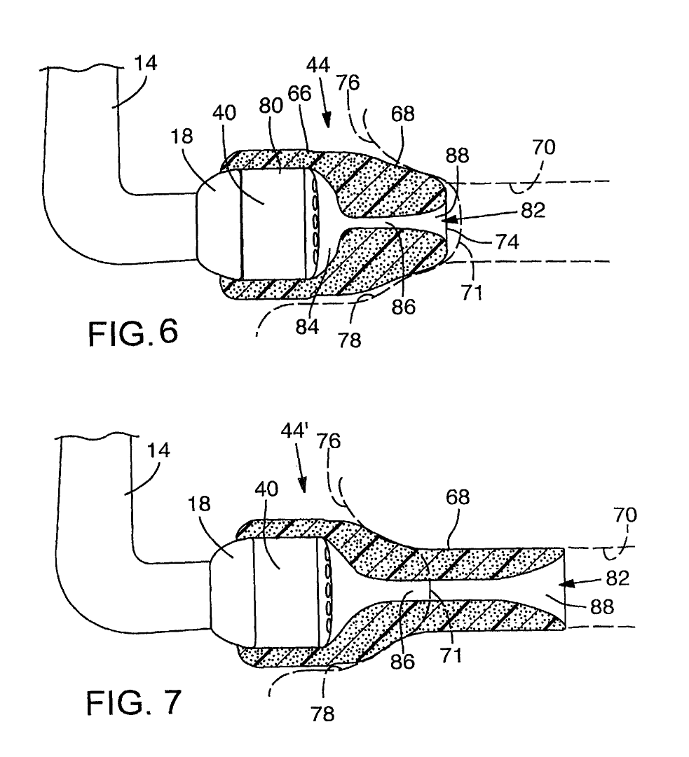

Returning to FIG. 1, the device further includes audio speakers 40, 42 (see FIGS. 6 and 7) attached to the respective end portions 18, 20 of the band 12. In FIG. 1, the audio speakers 40, 42 are at least partially concealed beneath hearing protectors 44, 46. Generally, the audio speakers 40, 42 are small, miniature speakers, such as those used in audio headphones well known in the art. The audio speakers 40, 42 may be bonded or molded onto the end portion 18, 20 or connected to the end portions with an adhesive or other suitable means. The audio speakers 40, 42 are electrically connected to the audio transmitter 22 through wires or other conductors (not shown) extending along the respective legs 14, 16. The wires may be positioned in a channel molded into the legs 14, 16 of the band 12 such that the wires are integrated into the legs.

Hearing protectors 44, 46 are attached to each of the respective speakers 40, 42 and engage the ears of the user. As more fully described below, the hearing protectors 44, 46 may be constructed of a compressible, deformable material (e.g., a suitable foam, silicon rubber, a hypoallergenic rubber compound, etc.). Each of the hearing protectors 44, 46 includes a speaker chamber 80 within which the speakers 40, 42 are positioned. The hearing protectors 44, 46 may also include a sound channel 82 that directs sound generated by the speakers 40, 42 into the ear canal of the user. The hearing protectors 44, 46 may be held against the walls of the user's ears by an inwardly directed force (shown by arrows A and B) that is created by the flexibility and resiliency of the band. The pressure required to sufficiently engage the hearing protectors with the ears of the user, and thus the resiliency of the band, may depend on the type of hearing protector attached to the speakers 40, 42. For instance, as discussed below, the hearing protectors 44, 46 may be supra-aural-type hearing protectors that do not enter the ear canal, and thus require some pressure to be held against the user's ears. On the other hand, the hearing protectors 44, 46 may be semi- or inner-aural-type hearing protectors that do enter the ear canal and require less pressure to be sufficiently engaged with the user's ears.

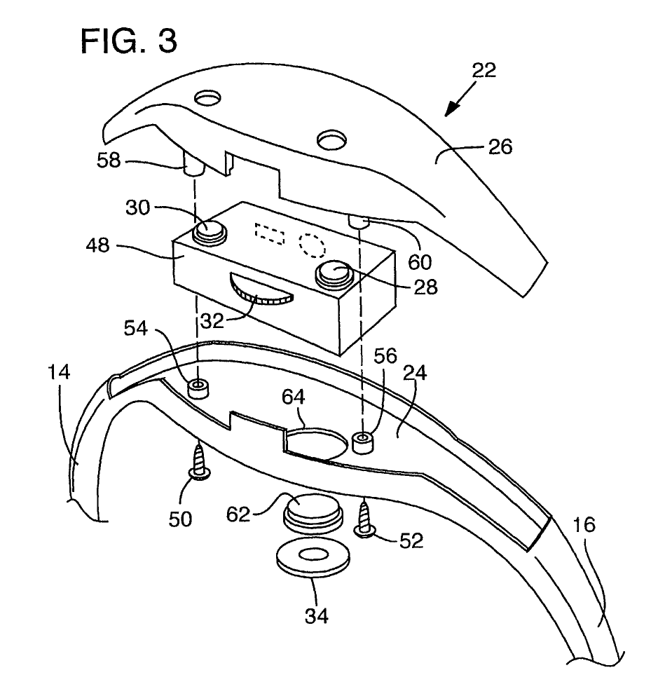

FIG. 3 shows the construction of the audio transmitter 22 of the representative implementation in greater detail. As noted above, the band includes the two legs 14, 16 and the support portion 24. The cover portion 26 attaches to the support portion 24 and forms at least a partial enclosure between the support portion and the cover portion. As shown in FIG. 3, the partial enclosure may form a low-profile housing having curved side surfaces, a curved outer surface, and a generally flat inner surface. Internal circuitry 48 of the audio transmitter 22 may be positioned within the enclosure. The shape of the internal circuitry 48 shown in FIG. 3 is for illustrative purposes only and is not intended to be limiting in any way. The internal circuitry 48 may have a variety of different shapes.

As shown in FIG. 3, the cover portion 26 may be fastened to the support portion 24 using threaded screws 50, 52 that extend through apertures 54, 56 and engage threaded receivers 58, 60. Although only two screws 50, 52 are shown in FIG. 3, the number and relative position of the screws may vary. Further, the cover portion 26 may be attached to the support portion 24 by other types of fasteners (e.g., adhesives, pegs, friction, etc.). A power source 62 for the audio transmitter 22 is also shown in FIG. 3. The power source 62 may comprise a flat disc-shaped battery or other suitable power source. In the illustrated implementation, the power source 62 may be accessed from the support portion 24 via the battery cover 34. The battery cover 34 may be threaded to engage a threaded aperture 64 in the support portion 24.

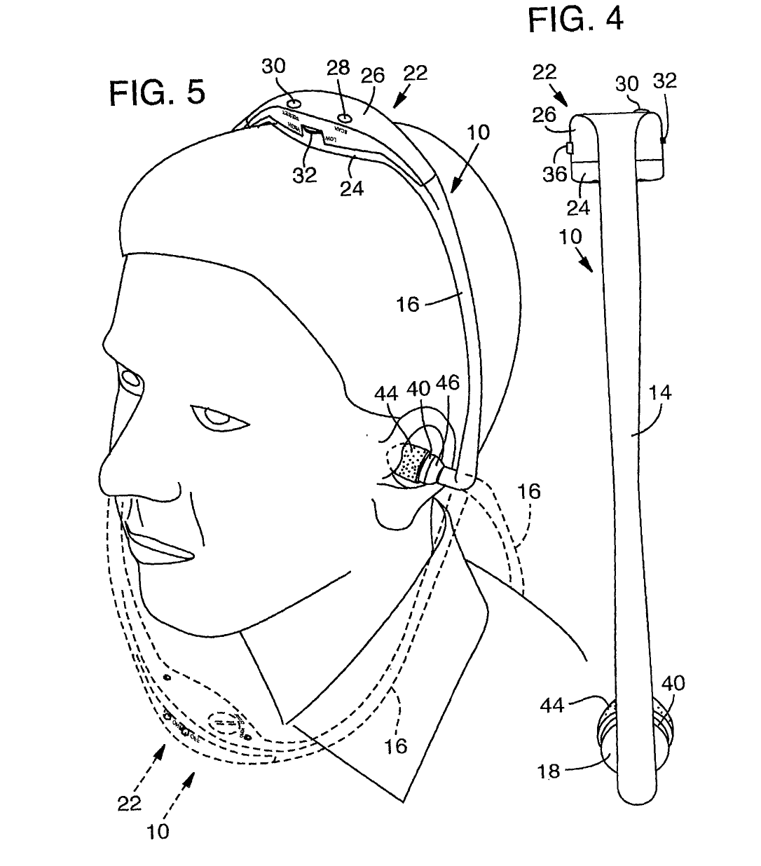

FIG. 4 shows a side view of a representative implementation of the personal audio device 10. The leg 14 is shown extending from the audio transmitter 22 and connecting with the speaker 40. As illustrated in FIG. 4, the leg 14 may smoothly taper from the dimensions of the transmitter 22 and have a thin profile relative to the user's head (e.g., 1-4 cm). The leg 14 may also be rounded or have smooth edges in order to provide added comfort to the user and flexibility in how the device 10 may be positioned.

FIG. 5 shows a user wearing the personal audio device 10. FIG. 5 illustrates several positions in which the device 10 may be worn by the user, including an over-the-head position (shown in solid lines) and two alternative positions (shown in dashed lines). For instance, the device 10 may be worn around the front or back of the user's neck. Although only three positions are shown in FIG. 5, these positions are not limited and other positions are possible.

The flexible and lightweight design of the device allows it to be worn comfortably by a user for an extended period of time. Further, because all of the components of the device are integrated into a single band, a user wearing the device may continue to operate in his full range of motion. Moreover, the simple controls on the audio transmitter allow a user wearing the device to easily adjust the audio transmission. Finally, the low-cost construction of the device makes it attractive to a large number of consumers (e.g., spectators at an autorace, industrial workers, etc.).

FIG. 6 shows a first representative implementation of the hearing protector 44 that may be used with the personal audio device 10. The hearing protector 44 is generally constructed of resilient foam composition well known in the art. For instance, any of the foams disclosed in U.S. Pat. Nos. 5,792,998 and 4,158,087 are suitable for constructing the hearing protector 44. Alternatively, the hearing protector 44 may be constructed of a silicon rubber (e.g., medical-grade silicon rubber) or other hypoallergenic rubber compound. When worn by the user, the shape and resiliency of the hearing protector 44 attenuates external noises to a sound level of, for instance, 90 db or less. The hearing protector 44 may be constructed using a variety of techniques well known in the art (e.g., injection molding, rotational molding, slush molding, dip molding, etc.).

In the illustrated implementation, the hearing protector 44 has a cylindrical main body portion 66 and an adjoining frustoconical tapered portion 68. The main body portion 66 typically has a greater diameter than the tapered portion 68, which tapers to a lesser diameter at a distal end of the hearing protector 44. The hearing protector 44 shown in FIG. 6 is termed a “supra-aural” hearing protector because the tapered portion 68 is designed to fit around the outer portion of the ear canal 70 so that an end portion 74 is adjacent to, but does not enter, the entrance 71 of the ear canal. When supported on the band 12, the shape and resiliency of the band holds the end portion 74 so that it is adjacent to the entrance 71 of the ear canal 70 and presses the tapered portion 68 against the walls 76, 78 surrounding the ear canal (i.e., the band presses the tapered portion against the concha of the ear). The deformable material of which the hearing protector 44 is made then deforms against the walls 76, 78 so that outside noises are effectively blocked.

In the illustrated implementation, the hearing protector 44 defines an interior speaker chamber 80 configured to receive the speaker 40 and part of the end portion 18. In another implementation, the speaker chamber 80 may be configured to extend only partially over the speaker 40 (as illustrated in FIGS. 4 and 5). The speaker chamber 80 has a cylindrical shape and begins at an end of the hearing protector 44 and extends along the central axis of the main body portion 66. In one particular implementation, the speaker chamber 80 has a sufficient diameter to frictionally engage the hearing protector 44 to the speaker 40 and part of the end portion 18. In this implementation, the speaker chamber 80 deforms slightly to fit the speaker 40. The speaker chamber 80 may also include a flanged portion at the end of the hearing protector 44 to enable better contact with the tapered surface of the end portion 18. The speaker chamber 80 may further include a shoulder portion in the interior of the chamber in order to properly align the speaker with the sound channel 82. Because the hearing protector 44 may be made from a flexible material that allows the hearing protector to be deformed, it may be easily removed by the user and interchanged with another hearing protector. Alternatively, the hearing protector 44 may be bound to the speaker 40 in any other suitable manner (e.g., an adhesive).

As shown in FIG. 6, the hearing protector 44 also defines a sound channel 82 extending axially from and in communication with the speaker chamber 80. The sound channel 82 is configured to direct sound generated from the speaker 40 into the ear canal 70 of the user. The sound channel may have a Venturi shape (i.e., a passage with a narrow throat). In one implementation, for instance, the sound channel 82 includes a sound-expansion-chamber portion 84, a central-chamber portion 86, and an end-chamber portion 88. The sound-expansion-chamber portion 84 is a frustoconical chamber located adjacent the speaker chamber 80 and extends along the central axis of the main body portion 66. The sound-expansion-chamber portion 84 has a sufficient diameter and width to allow sound waves generated by the full diameter of the speaker 40 to travel to the central-chamber portion 86. Thus, sound waves from the speaker 40 are not blocked. Although the sound-expansion-chamber portion 84 shown in FIG. 6 has a frustoconical shape, other shapes may be used to permit adequate sound transmission (e.g., a dish shape, a spherical shape, etc.). The central-chamber portion 86 is a cylindrical chamber along the central axis of the main body portion 66 and the tapered portion 68. The central-chamber portion 86 extends from the sound-expansion-chamber portion 84 to the end-chamber portion 88 and has a diameter smaller than that of the sound-expansion-chamber portion 84. The narrow central-chamber portion 86 allows for the transmission of sound waves generated by the speaker 40 without compromising the noise attenuating benefits of the hearing protector 44. The end-chamber portion 88 is a frustoconical chamber adjacent the central chamber 86 and extends along the central axis of the tapered portion 68 to the end portion 74. The diameter of the end-chamber portion 88 at the end portion 74 is larger than that of the central-chamber portion 86 and, as shown in FIG. 6, may have a flared or trumpet shape. The larger diameter at the end portion 74 prevents the sound channel 82 from being pinched shut when the hearing protectors are worn.

FIG. 7 shows a second representative implementation of a hearing protector 44′. The hearing protector 44′ is similar to the first representative implementation except that the tapered portion 68 is elongated and configured to fit inside the ear canal 70 during use. The hearing protector 44′ is an “inner-aural” hearing protector because it extends beyond the entrance 71 of the ear canal 70 and fits substantially within the ear canal. In another implementation, the hearing protector 44′ may be a “semi-aural” hearing protector that only slightly enters the ear canal 70. The hearing protector 44′ is made of a material similar to the material used in the hearing protector 44. The sound channel 82 of this implementation further includes an elongated central-chamber portion 86 that transmits sound from the speaker 40 to the end-chamber portion 88. The diameter of the central-chamber portion 86 in this implementation may be larger than the diameter of the central-chamber portion in hearing protector 44. The larger diameter of the central-chamber portion 86 prevents the central-chamber portion from being pinched shut when worn by the user. In use, the hearing protector 44′ is first compressed by the user and fit into the ear canal 70. The hearing protector 44′ then expands toward its original shape to seal the ear canal 70 from external sounds.

Having illustrated and described the principles of the illustrated implementations, it will be apparent to those skilled in the art that the implementations can be modified in arrangement and detail without departing from such principles.

For instance, in one alternative implementation, the legs 14, 16 do not make a completed band 12, but are instead connected separately to the audio transmitter 22. In this implementation, the audio transmitter 22 is molded to include sleeves at each end that can receive the ends of the legs 14, 16, which are held in place through an adhesive.

In an alternative implementation of the device, an additional insulator is disposed adjacent the speaker chamber 80 of the hearing protector 44. The additional insulator further seals the sound chamber 82 from external sound.

In yet another implementation of the device, a sunglass frame or protective eye guard may be affixed to the band 12. In this implementation, the user wears the device as he would normally wear sunglasses or protective eyewear, but inserts the ends of the headband into his ears.

In still another implementation, the hearing protectors 44, 46 of the device do not include a sound channel 82. Thus, the device functions as a conventional band earplug.

In view of the many possible implementations, it will be recognized that the illustrated implementations include only examples and should not be taken as a limitation on the scope of the disclosed technology. Rather, the disclosed technology is defined by the following claims. I therefore claim all such implementations that come within the scope of these claims.

What is claimed is:

1. A personal audio device, comprising:
a flexible band configured to extend at least partially around a head of a user, the band having a first leg and a second leg, the first and second legs terminating at respective first and second end portions;
an audio transmitter coupled to the band;
first and second audio speakers attached to the respective first and second end portions of the band, the first and second audio speakers being electrically connected to the audio transmitter; and
first and second hearing protectors mounted on the respective first and second audio speakers, each of the hearing protectors having a main body portion and a tapered portion configured to fit an ear of the user and attenuate external sound, each hearing protector having an internal speaker chamber defined therein within which one of the audio speakers is received and an adjacent sound channel extending through the tapered portion and configured to transmit sound from the respective audio speaker to the ear of the user.

2. The device of claim 1, wherein the band has sufficient resiliency to urge the first and second hearing protectors against the ears of the user to at least partially engage the hearing protectors against or in the ear canals of the user, thereby attenuating external sound.

3. The device of claim 2, wherein when the device is worn by the user, the hearing protectors attenuate external sound relative to the user to a sound level of 90 db or below.

4. The device of claim 1, wherein the hearing protectors are made of a compressible foam.

5. The device of claim 1, wherein the hearing protectors are made of silicon rubber.

6. The device of claim 1, wherein the sound channel has a Venturi shape.

7. The device of claim 1, wherein the sound channel comprises:
a frustoconical sound-expansion-chamber portion adjacent the speaker chamber, the sound-expansion-chamber portion extending away from the respective audio speaker;
a cylindrical central-chamber portion adjacent the sound-expansion-chamber portion, the central-chamber portion having a diameter smaller than a diameter of the sound-expansion-chamber portion, the central-chamber portion extending through the main body portion into the tapered portion; and
a frustoconical end-chamber portion adjacent the central-chamber portion and extending to an end of the tapered portion, the end-chamber portion having a diameter larger than the diameter of the central-chamber portion.

8. The device of claim 7, wherein the end-chamber portion has a trumpet shape.

9. The device of claim 1, wherein each hearing protector is removable from the respective audio speaker.

10. The device of claim 1, wherein the tapered portion is configured to contact a portion of the ear adjacent the ear canal without entering the ear canal.

11. The device of claim 1, wherein the tapered portion is configured to extend at least partially into the ear canal of the user after insertion.

12. The device of claim 1, wherein the audio transmitter is located at a position on the band approximately midway between the end portions.

13. The device of claim 1, wherein the audio transmitter further comprises a jack configured to input electrical signals from an external device.

14. The device of claim 1, wherein the audio transmitter is a radio positioned in a housing on the band, the audio transmitter further including at least one tuning button that protrudes through the housing, thereby allowing the user to tune the radio while the device is worn.

15. A personal audio device, comprising:
a flexible band configured to extend at least partially around a head of a user, the band having first and second end portions located at opposite ends of the band;
an audio transmitter coupled to the band and positioned between the ends of the band;
first and second audio speakers attached to respective end portions of the band, the first and second audio speakers being electrically connected to the audio transmitter; and
first and second hearing protectors removably mounted on the respective first and second audio speakers, the hearing protectors being configured to attenuate external noise, each of the hearing protectors further having a Venturi-shaped sound channel configured to transmit sound from the respective audio speaker to an ear of the user.

16. The device of claim 15, wherein each of the hearing protectors comprises:
a main body portion;
a tapered portion adjacent the main body portion; and
a speaker chamber defined in the main body portion within which one of the audio speakers is received.

17. The device of claim 16, wherein the tapered portion is configured to contact a portion of the ear adjacent the ear canal without entering the ear canal.

18. The device of claim 16, wherein the tapered portion is configured to extend at least partially into the ear canal of the user after insertion.

19. The device of claim 16, wherein the Venturi-shaped sound channel comprises:
a frustoconical sound-expansion-chamber portion adjacent the speaker chamber, the sound-expansion-chamber portion extending away from the respective audio speaker;
a cylindrical central-chamber portion adjacent the sound-expansion-chamber portion, the central-chamber portion having a diameter smaller than a diameter of the sound-expansion-chamber portion, the central-chamber portion extending through the main body portion into the tapered portion; and
a frustoconical end-chamber portion adjacent the central-chamber portion and extending to an end of the tapered portion, the end-chamber portion having a diameter larger than the diameter of the central-chamber portion.

20. The device of claim 19, wherein the end-chamber portion has a trumpet shape.

21. The device of claim 15, wherein the hearing protectors are made of a compressible foam.

22. The device of claim 15, wherein the hearing protectors are made of silicon rubber.

23. The device of claim 15, wherein the band has sufficient resiliency to urge the first and second hearing protectors against the ears of the user to at least partially engage the hearing protectors against or in the ear canals of the user, thereby attenuating external sound.

24. The device of claim 23, wherein when the device is worn by the user, the hearing protectors attenuate external sound relative to the user to a sound level of 90 db or below.

25. The device of claim 15, wherein the audio transmitter is located at a position on the band approximately midway between the end portions.

26. The device of claim 15, wherein the audio transmitter further comprises a jack configured to input electrical signals from an external device.

27. The device of claim 15, wherein the audio transmitter is a radio positioned in a housing on the band, the audio transmitter further including at least one tuning button that protrudes through the housing, thereby allowing the user to tune the radio while the device is worn.

28. A hearing protector for use with a personal audio device, comprising:
a main body portion having a cylindrical shape;
a tapered portion integrally attached to the main body portion and configured to engage an ear of the user;
a speaker chamber formed in the main body portion, the speaker chamber being configured to receive an audio speaker;
a sound expansion chamber adjacent the speaker chamber, the sound expansion chamber having a tapered, cylindrical shape and extending toward the tapered portion;
a central chamber adjacent the sound expansion chamber, the central chamber having a cylindrical shape and having a diameter smaller than a diameter of the sound expansion chamber, the central chamber extending from the main body portion into the tapered portion; and
an end chamber adjacent the central chamber and extending to an end of the tapered portion, the end chamber having a cylindrical shape and having a diameter larger than the diameter of the central channel.

29. The hearing protector of claim 28, wherein the end chamber has a trumpet shape as it extends to the end of the tapered portion.

30. The device of claim 28, wherein the tapered portion is configured to contact a portion of the ear adjacent the ear canal without entering the ear canal.

31. The device of claim 28, wherein the tapered portion is configured to extend at least partially into the ear canal of the user after insertion.

32. The device of claim 28, wherein the hearing protectors are made of a compressible foam.

33. The device of claim 28, wherein the hearing protectors are made of silicon rubber.

34. A dual-use head-worn device providing hearing protection and allowing a user to listen to desired audio transmissions from a source within the device, the device comprising:
a resilient generally C-shaped member having opposed ends with respective ear interface members, each ear interface member having an audio speaker and a resilient hearing protector at least partially covering the audio speaker, the hearing protector providing the user with hearing protection by attenuating ambient sounds and having a sound channel defined therein to convey sounds from the audio speaker to the user's ear when the device is worn; and
a low-profile housing coupled to the band at a position between the ends of the band, the housing having a first pair of generally opposite curved side surfaces and second adjacent pair of surfaces that includes a curved outer surface and an opposite inner surface with a generally flat portion.

35. The device of claim 34, further comprising an audio transmitter positioned in the housing and electrically connected to each audio speaker, the audio transmitter having tactile controls extending through openings in the housing allowing the user to selectively control sounds from the transmitter while the device is worn.

36. The device of claim 34, wherein the low-profile housing allows the device to be worn in multiple positions while the ear interface members are engaged with the ears, including over the user's head, forward of the user's head, and rearward of the user's head.

37. The device of claim 34, wherein the housing is located approximately midway between the opposed ends of the band.

38. The device of claim 34, wherein the sound channel comprises:
a frustoconical sound-expansion-chamber portion adjacent the speaker chamber, the sound-expansion-chamber portion extending away from the respective audio speaker;
a cylindrical central-chamber portion adjacent the sound-expansion-chamber portion, the central-chamber portion having a diameter smaller than a diameter of the sound-expansion-chamber portion, the central-chamber portion extending through the main body portion into the tapered portion; and
a frustoconical end-chamber portion adjacent the central-chamber portion and extending to an end of the tapered portion, the end-chamber portion having a diameter larger than the diameter of the central-chamber portion.

39. The device of claim 38, wherein the end-chamber portion has a trumpet shape.
